(19) United States
(12) Patent Application Publication
(10) Pub. No.: US 2021/0038197 A1
(43) Pub. Date: Feb. 11, 2021

(54) ULTRASOUND IMAGING METHOD AND ULTRASOUND IMAGING DEVICE

(71) Applicant: SHENZHEN MINDRAY BIO-MEDICAL ELECTRONICS CO., LTD., Shenzhen (CN)

(72) Inventors: Jie LIU, SHENZHEN (CN); Jianguang ZHU, SHENZHEN (CN); Lei LI, SHENZHEN (CN); Qingpeng LI, SHENZHEN (CN); Xujin HE, SHENZHEN (CN); Yaoxian ZOU, SHENZHEN (CN)

(21) Appl. No.: 17/079,274

(22) Filed: Oct. 23, 2020

Related U.S. Application Data
(63) Continuation of application No. PCT/CN2018/084413, filed on Apr. 25, 2018.

(51) Int. Cl. A61B 8/08; A61B 8/12; A61B 8/00

(52) U.S. Cl. CPC A61B 8/5207; A61B 8/12; A61B 2562/0223; A61B 8/5246; A61B 8/461

(57) ABSTRACT

An ultrasound imaging method and an ultrasound imaging device are provided. The ultrasound imaging device includes a probe, a transmitting circuit, a receiving circuit and a processor. The ultrasound imaging method include: obtaining the position information of an interventional object inserted into a target object; determining the optimized imaging parameters according to the position information; transmitting, at least one first angle, the first ultrasound wave to the interventional object according to the optimized imaging parameters, and receiving the first ultrasound echo returned from the interventional object to obtain the first ultrasound echo data; generating the ultrasound image of the interventional object according to the first ultrasound echo data; and obtaining the ultrasound image of the target object, and combining the ultrasound image of the target object with the ultrasound image of the interventional object to obtain the combined image.

### CROSS-REFERENCE TO RELATED APPLICATION

This application is a continuation application of International Patent Application No. PCT/CN2018/084413, filed with the China National Intellectual Property Administration (CNIPA) of People's Republic of China on Apr. 25, 2018, and entitled “ULTRASOUND IMAGING METHOD AND ULTRASOUND IMAGING DEVICE”. The entire content of the above-identified applications is incorporated herein by reference.

### TECHNICAL FIELD

The present disclosure relates to medical devices, in particular to an ultrasound imaging method and ultrasound imaging device.

### BACKGROUND

In clinical practice, real-time ultrasound imaging has been widely used to guide puncture needles. Doctors can perform the puncture in reference to the puncture needle images, which can effectively improve the success rate of the puncture operation and reduce the trauma.

Due to the influence of different factors such as patient size, puncture position, operation method, etc., the obtained puncture needle image is most likely not at the optimal state. Therefore, it is desired to optimize the puncture needle image to make the puncture needle image to be displayed more clearly and accurately to the operator.

However, in the actual process, the operator needs to manually adjust a series of parameters to optimize the puncture needle image, which not only requires the operator to be familiar with the machine, but also increases the operation steps to the operator during the operation, which reduces the operation efficiency.

### SUMMARY

The embodiments of the present disclosure provide ultrasound imaging methods and ultrasound imaging devices for improving the operation efficiency.

In one embodiment, an ultrasound imaging method is provided, which may include: obtaining a position information of an interventional object inserted into a target object; determining an optimized imaging parameter according to the position information; transmitting a first ultrasound wave to the interventional object in at least one first angle according to the optimized imaging parameter, and receiving a first ultrasound echo returned from the interventional object to obtain a first ultrasound echo data; generating an ultrasound image of the interventional object according to the first ultrasound echo data; and obtaining an ultrasound image of the target object, and combining the ultrasound image of the target object with the ultrasound image of the interventional object to obtain a combined image.

In one embodiment, an ultrasound imaging method is provided, which may include: transmitting a first ultrasound wave to an interventional object inserted into a target object in at least one first angle according to a first imaging parameter, and receiving a first ultrasound echo returned from the interventional object to obtain a first ultrasound echo data; generating a first ultrasound image of the interventional object according to the first ultrasound echo data; receiving a first operation instruction; determining a second imaging parameter according to the first operation instruction; transmitting a second ultrasound wave to the interventional object in the at least one first angle according to the second imaging parameter, and receiving a second ultrasound echo returned from the interventional object to obtain a second ultrasound echo data; generating a second ultrasound image of the interventional object according to the second ultrasound echo data; and obtaining an ultrasound image of the target object, and combining the ultrasound image of the target object with the second ultrasound image of the interventional object to obtain a combined image.

In one embodiment, an ultrasound imaging device is provided, which may include: a processor that is configured to obtain a position information of an interventional object inserted into a target object and determine an optimized imaging parameter according to the position information; a probe; a transmitting circuit that is configured to excite the probe to transmit a first ultrasound wave to the interventional object in at least one first angle according to the optimized imaging parameter; and a receiving circuit that is configured to control the probe to receive a first ultrasound echo returned from the interventional object to obtain a first ultrasound echo data; where the processor is further configured to generate an ultrasound image of the interventional object according to the first ultrasound echo data, obtain an ultrasound image of the target object, and combine the ultrasound image of the target object with the ultrasound image of the interventional object to obtain a combined image.

In one embodiment, an ultrasound imaging device is provided, which may include: a probe; a transmitting circuit that is configured to excite the probe to transmit a first ultrasound wave to an interventional object inserted into a target object in at least one first angle according to a first imaging parameter; a receiving circuit that is configured to control the probe to receive a first ultrasound echo returned from the interventional object to obtain a first ultrasound echo data; and a processor that is configured to generate a first ultrasound image of the interventional object according to the first ultrasound echo data; where, the processor is further configured to receive a first operation instruction and determine a second imaging parameter according to the first operation instruction; the transmitting circuit is further configured to excite the probe to transmit a second ultrasound wave to the interventional object in the at least one first angle according to the second imaging parameter; the receiving circuit is further configured to control the probe to receive a second ultrasound echo returned from the interventional object to obtain a second ultrasound echo data; and the processor is further configured to generate a second ultrasound image of the interventional object according to the second ultrasound echo data, obtain an ultrasound image of the target object, and combine the ultrasound image of the target object with the second ultrasound image of the interventional object to obtain a combined image.

In one embodiment, a computer readable storage medium is provided, which may store instructions. When being executed by a computer, the instructions cause the computer to perform the ultrasound imaging methods above.

It can be seen that in the technical solutions of the embodiments of the present disclosure, after obtaining the position information of the interventional object, the optimized imaging parameter may be determined according to the position information, the first ultrasound waves may be transmitted to the interventional object according to the optimized imaging parameters to obtain the first ultrasound echo data to generate the ultrasound image of the interventional object, and the ultrasound image of the interventional object and the ultrasound image of the target object may be combined to obtain the combined image. Therefore, it is not necessary for the operator to adjust the parameters manually to optimize the ultrasound image, thereby solving the problem of low operation efficiency and improving the operating efficiency.

### BRIEF DESCRIPTION OF THE DRAWINGS

FIG. 1 is a schematic block diagram of an ultrasound imaging device in one embodiment;

FIG. 2 is a flowchart of an ultrasound imaging method in one embodiment;

FIG. 3 is a schematic diagram of a probe in one embodiment;

FIG. 4 is a schematic diagram of the initial displaying of a puncture needle in one embodiment;

FIG. 5 is a schematic diagram of a displaying of the puncture needle after the adjustment in one embodiment;

FIG. 6 is a schematic diagram of another initial displaying of the puncture needle in one embodiment;

FIG. 7 is a schematic diagram of another displaying of the puncture needle after the adjustment in one embodiment;

FIG. 8 is a schematic diagram of an initial displaying of a focus in one embodiment;

FIG. 9 is a schematic diagram of the focus adjustment in one embodiment;

FIG. 10 is a schematic diagram of the reflection of the ultrasound waves in one embodiment;

FIG. 11 is a schematic diagram of another reflection of the ultrasound waves in one embodiment;

FIG. 12 is a schematic diagram of another reflection of the ultrasound waves in one embodiment;

FIG. 13 is a schematic diagram of the image combination based on wavelet transformation in one embodiment; and

FIG. 14 is a schematic flowchart of another ultrasound imaging method in one embodiment.

### DETAILED DESCRIPTION

The embodiments of the present disclosure provide ultrasound imaging methods and ultrasound imaging devices for improving operation efficiency.

The terms “first”, “second”, “third”, “fourth”, etc. (if any) in the specification, claims and drawings of the present disclosure are used to distinguish similar objects, but not describe a specific order or sequence. It should be understood that the data described in this way can be interchanged under appropriate circumstances such that the embodiments described herein can be implemented in an order other than what are illustrated or described herein. In addition, the terms “including” and “having” and any variations thereof are intended to mean non-exclusive inclusions. For example, a process, method, system, product or device that includes a series of steps or units is not necessarily limited to the listed steps or units, but may include other steps or units that are not listed or are inherent to the process, method, product or device.

FIG. 1 is a schematic block diagram of an ultrasound imaging device 10 in one embodiment. The ultrasound imaging device 10 may include a probe 100, a transmitting circuit 101, a transmitting/receiving switch 102, a receiving circuit 103, a beam forming circuit 104, a processor 105 and a display 106. The transmitting circuit 101 may excite the probe 100 to transmit ultrasound waves to a target object. The receiving circuit 103 may receive the ultrasound echoes returned from the target object through the probe 100, thereby obtaining the ultrasound echo signal/data. The ultrasound echo signal/data may be sent to the processor 105 after the beam forming processing is performed thereon by the beam forming circuit 104. The processor 105 may process the ultrasound echo signal/data to obtain the ultrasound image of the target object or the ultrasound image of the interventional object. The ultrasound images obtained by the processor 105 may be stored in the memory 107. These ultrasound images may be displayed on the display 106.

In one embodiment, the display 106 of the ultrasound imaging device 10 may be a touch screen, a liquid crystal display screen, etc., or may be an independent display device such as a liquid crystal display or a TV independent from the ultrasound imaging device 10. It may also be the display screen on an electronic device such as a mobile phone, a tablet computer, or the like.

In one embodiment, the memory 107 of the ultrasound imaging device 10 may be a flash memory card, a solid-state memory, a hard disk, or the like.

In one embodiment of the present disclosure, a computer-readable storage medium may also be provided. The computer-readable storage medium may store multiple program instructions. After being called and executed by the processor 105, the multiple program instructions can perform part or all or any combination of the steps in the ultrasound imaging methods in the embodiments.

In one embodiment, the computer-readable storage medium may be the memory 107, which may be a non-volatile storage medium such as a flash memory card, a solid-state memory, a hard disk, or the like.

In one embodiment of the present disclosure, the processor 105 of the ultrasound imaging device 10 may be implemented by software, hardware, firmware, or a combination thereof, and may use circuits, single or multiple application specific integrated circuits (ASIC), single or multiple general-purpose integrated circuits, single or multiple microprocessors, single or multiple programmable logic devices, or a combination of the foregoing circuits or devices, or other suitable circuits or devices, such that the processor 105 can perform the steps of the ultrasound imaging methods in the embodiments of the present disclosure.

The ultrasound imaging methods will be described in detail below.

It should be noted that, with reference to the schematic block diagram of the ultrasound imaging device 10 shown in FIG. 1, the ultrasound imaging method provided in the embodiments of the present disclosure may be applied to the following application, where the operator may place the probe 100 on the body surface of the site to be punctured and insert the puncture needle from the side of the probe 100, and may, through the display 106, observe the tissue and the positions of the puncture needle and the needle tip in the tissue. Therefore, the operator can clearly know the path of the puncture needle and the position to be reached. In this way, under the guidance of the image, the operator can perform the puncture operation more intuitively and efficiently.

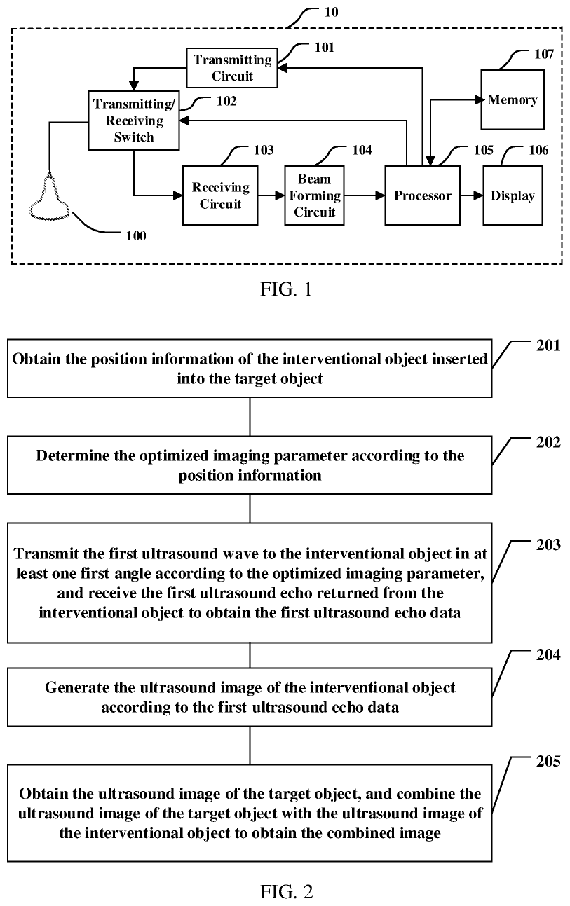

Referring to FIG. 2, in one embodiment, an ultrasound imaging method is provided, which may be applied to the ultrasound imaging device 10. The ultrasound imaging method may include the following steps.

In step 201, the position information of an interventional object inserted into a target object may be obtained.

In this embodiment, the processor 105 may obtain the position information of an interventional object inserted into a target object, and determine the optimized imaging parameters according to the position information.

In a clinical operation, when the interventional object is inserted into the target object, the ultrasound imaging device 10 may position the interventional object to obtain the position information of the interventional object.

For ease of description, in the embodiments of the present disclosure, the puncture needle is taken as an example of the interventional object. Correspondingly, the position information of the interventional object may include the position of the needle tip of the puncture needle. In practical applications, the interventional object may be other objects, which will not be limited here.

It should be noted that in practical applications, there are many ways to obtain the position information of the interventional object, including positioning technology through magnetic field induction, image pattern recognition technology, infrared or laser technology, etc., which will not be limited here.

In one embodiment, the position information of the interventional object may be obtained by magnetic field induction positioning technology. The obtaining the position information of the interventional object inserted into the target object may include the processor 105 detecting the magnetic induction intensity generated by the magnetized puncture needle and determining the position of the needle tip of the puncture needle according to the magnetic induction intensity.

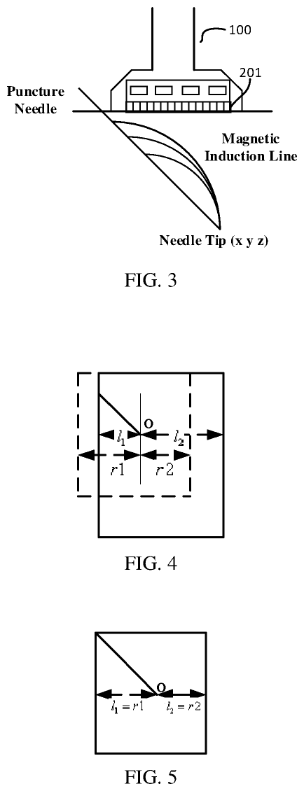

The magnetic field induction positioning technology can be understood as a technology that uses the penetration of a magnetic field to unshielded objects to achieve real-time positioning in a non-visible state. Exemplarily, the process of determining the position of the needle tip of the puncture needle based on the magnetic field induction positioning technology may include the following step. The operator may magnetize the puncture needle through the magnetization cylinder to obtain the magnetized puncture needle. When the magnetized puncture needle is close to the probe 100 of the ultrasound imaging device 10, since the magnetized puncture needle generates a magnetic field and the probe 100 is provided with a magnetic field sensor 201 formed by magnetically sensitive materials, the magnetized puncture needle will affect the magnetic field around the magnetic field sensor 201, as shown in FIG. 3. Therefore, the magnetic field sensor may detect the magnetic induction intensity of the magnetic field generated by the puncture needle, and the ultrasound imaging device 10 may determine the change of the magnetic field around the magnetic field sensor according to the change of the detected magnetic induction intensity, and calculate the coordinate information and orientation information of the needle tip of the puncture needle in real time according to the change of the magnetic field, so as to determine the positon of the needle tip of the puncture needle.

In one embodiment, the position information of the interventional object may be obtained through image pattern recognition technology. For example, after the puncture needle is inserted into the target object, the ultrasound imaging device 10 may transmit ultrasound waves through the probe 100 to obtain a B-mode ultrasound image (hereinafter referred to as B-mode image) that represents the puncture needle and the tissue, perform image enhancement and equalization processing on the B-mode image, and determine the position of the needle tip of the puncture needle in the B-mode image through image pattern recognition.

In one embodiment, the position information of the interventional object may be obtained by infrared or laser technology. For example, the depth and displacement or the like of the interventional object may be detected by infrared or laser so as to determine the position of the needle tip of the puncture needle in the ultrasound image.

In summary, in the embodiments of the present disclosure, there are many ways to position the interventional object, which will not be described in detail here.

It should be noted that in practical applications, the target object may be the face, spine, heart, uterus, or pelvic floor, etc., or other parts of human tissue, such as the brain, bones, liver, or kidneys, etc., which will not be limited here.

In step 202, the optimized imaging parameters may be determined according to the position information of the interventional object.

After obtaining the position information of the interventional object, the processor 105 may determine the optimized imaging parameters according to the position information of the interventional object, so as to transmit the first ultrasound waves to the interventional object according to the optimized imaging parameters. The optimized imaging parameters may include at least one of the scanning range of the ultrasound waves, the scanning depth of the ultrasound waves and the focus position of the ultrasound waves. The methods for determining the optimized imaging parameters will be described below.

In one embodiment, the scanning range of the imaging may be determined according to the position information of the interventional object. The processor 105 may determine the scanning range of the first ultrasound wave according to the position of the needle tip of the puncture needle such that the distance from the position of the needle tip of the puncture needle to the longitudinal boundary of the display area of the ultrasound image of the target object satisfies the first preset condition. Specifically, the distance from the position of the needle tip of the puncture needle to the longitudinal boundary of the display area of the ultrasound image of the target object may be determined. Referring to FIG. 4, which is a schematic diagram of the initial displaying of the puncture needle in one embodiment, the distances from the position of the needle tip of the puncture needle (shown as o in the figure) to the longitudinal boundaries of the display area of the ultrasound image are respectively l₁ and l₂. The first preset condition may be that l₁ is not greater than the first preset value r1 and l₂ is not greater than the second preset value r2. The ultrasound imaging device 10 may determine whether the distances from the position of the needle tip of the puncture needle to the longitudinal boundaries of the display area of the ultrasound image meet the first preset condition. If not, the scanning range of the first ultrasound waves may be adjusted such that the distances from the position of the needle tip of the puncture needle to the longitudinal boundaries of the display area of the ultrasound image meet the first preset condition. FIG. 5 is a schematic diagram of the displaying of the puncture needle after the adjustment, in which the distances from the position o of the needle tip of the puncture needle to the longitudinal boundaries of the display area of the ultrasound image are respectively l₁=r1 and l₂=r2. If the distances meet the first preset condition, the ultrasound imaging device 10 may determine the current scanning range of the ultrasound image of the target object as the optimized imaging parameter.

In one embodiment, the first preset condition may also be that l₁ is within the first preset interval [a, b] or l₂ is within the second preset interval [c, d], where a, b, c and d are all positive numbers. Therefore, the first preset condition will not be limited herein.

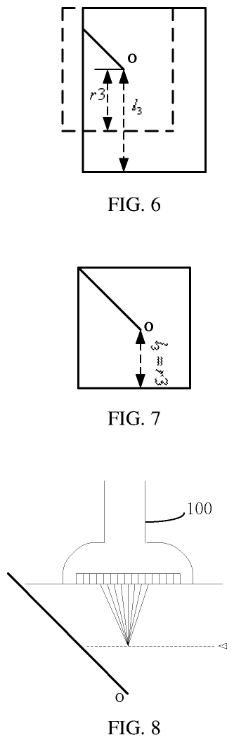

In one embodiment, the scanning depth of the imaging may be determined according to the position information of the interventional object. The processor 105 may determine the scanning depth of the first ultrasound waves according to the position of the needle tip of the puncture needle, such that the distance from the position of the needle tip of the puncture needle to the horizontal boundary of the display area of the ultrasound image of the target object satisfies the second preset condition. Specifically, the distance from the position of the needle tip of the puncture needle to the horizontal boundary of the display area of the ultrasound image of the target object may be determined. Referring to FIG. 6, which is a schematic diagram of another initial displaying of the puncture needle in one embodiment, the distance from the position o of the needle tip of the puncture needle to the horizontal boundary of the display area of the ultrasound image is l₃. The second preset condition may be that l₃ is not greater than the third preset value r3. The ultrasound imaging device 10 may determine whether the distance from the position of the needle tip of the puncture needle to the horizontal boundary of the display area of the ultrasound image meet the second preset condition. If not, the scanning depth of the first ultrasound waves may be adjusted such that the distance from the position of the needle tip of the puncture needle to the horizontal boundary of the display area of the ultrasound image meets the second preset condition. FIG. 7 is a schematic diagram of another displaying of the puncture needle after the adjustment, in which the distance from the position o of the needle tip of the puncture needle to the horizontal boundary of the display area of the ultrasound image is l₃=r3. If the distance meets the second preset condition, the ultrasound imaging device 10 may determine the current scanning depth of the ultrasound image of the target object as the optimized imaging parameter.

In one embodiment, the second preset condition may also be that l₃ is within the third preset interval [e, f], where e and f are both positive numbers. Therefore, the second preset condition will not be limited herein.

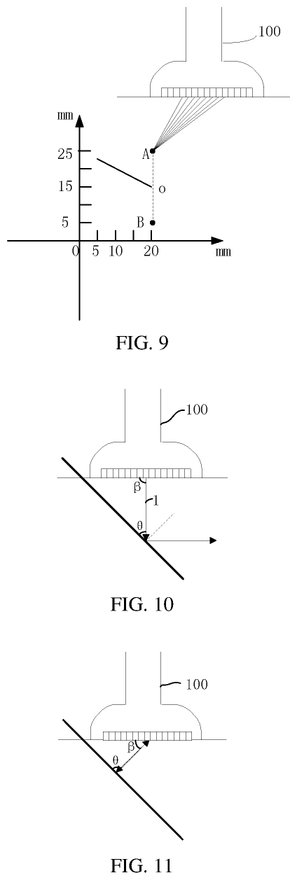

In one embodiment, the focus position of the ultrasound waves in the imaging may be determined according to the position information of the interventional object. The processor 105 may determine the focus position of the first ultrasound waves according to the position of the needle tip of the puncture needle, such that the needle tip of the puncture needle is within the range of the focus position of the first ultrasound waves. Referring to FIG. 8, which is a schematic diagram of an initial displaying of the focus in one embodiment, the position o of the needle tip of the puncture needle is not at the focus position of the ultrasound wave transmitted by the probe 100, resulting in that the needle tip of the puncture needle is blurry in the ultrasound image. Therefore, the ultrasound imaging device may determine whether the needle tip of the puncture needle is within the range of the focus of the ultrasound image of the target object, that is, whether it is at the focus of the current ultrasound waves. If not, the position of the focus may be adjusted, or the focusing range may be increased. For example, assuming that the coordinates of the needle tip o of the puncture needle are (20 mm, 15 mm), the coordinates of the focus are (20 mm, 25 mm), and the focus can be adjusted by a step of 10 mm, the ultrasound imaging device 10 may adjust the focus to (20 mm, 15 mm). Alternatively, if the focus can be adjusted by a step of 20 mm, referring to FIG. 9 that is a schematic diagram of a focus adjustment, the ultrasound imaging device 10 may add a focus B at (20 mm, 5 mm), such that the needle tip of the puncture needle is between the original focus A and the added focus B, thereby enable the needle tip of the puncture needle to be displayed more clearly.

In addition, in the case that the needle tip of the puncture needle is within the range of the focus, the ultrasound imaging device 10 may determine the current position of the focus of the ultrasound waves as the optimized imaging parameter.

In step 203, the first ultrasound waves may be transmitted to the interventional object in at least one first angle according to the optimized imaging parameter, and the first ultrasound echoes returned from the interventional object may be received to obtain the first ultrasound echo data.

In this embodiment, the probe 100 may be excited through the transmitting circuit 101 to transmit the first ultrasound waves to the interventional object in at least one first angle according to the optimized imaging parameters, and may be controlled through the receiving circuit 103 to receive the first ultrasound echoes returned from the interventional object to obtain the first ultrasound echo data.

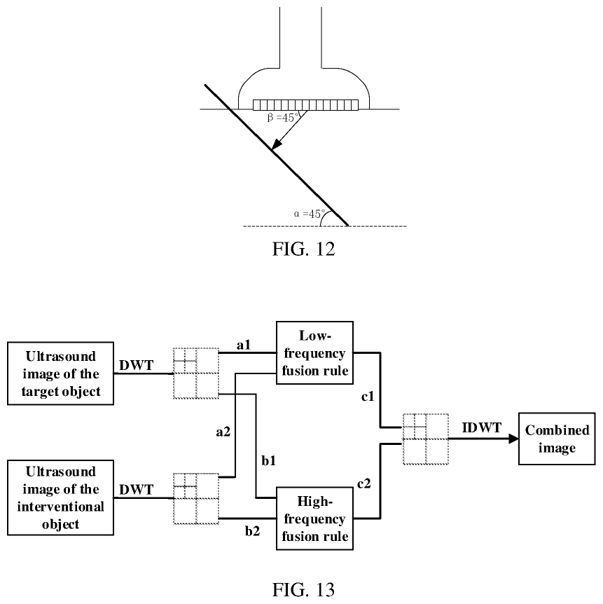

It should be noted that when performing the puncture operation, the puncture needle may be inserted into the tissue at a certain angle with respect to the surface of the probe. Due to the large acoustic impedance of the puncture needle, it is more difficult for the ultrasound waves to penetrate the puncture needle. The ultrasound echoes may be obtained to generate the image of the puncture needle. The first angle may be an angle favorable for receiving the ultrasound echoes returned from the interventional object obliquely inserted into the target object. Referring to FIG. 10 and FIG. 11 that are schematic diagrams of the reflection of the ultrasound waves in one embodiment, the angle θ is the angle between the ultrasound wave and the puncture needle, and the angle β is the angle in which the probe transmits the ultrasound waves. In FIG. 10, the probe may transmit the ultrasound wave 1 perpendicularly, that is, the angle β is 90°, and the angle θ is an acute angle. Therefore, the reflection direction of the ultrasound wave 1 on the surface of the puncture needle is not coincide with the transmitting direction of the ultrasound wave 1, that is, less ultrasound echoes can return to the probe, which lead to weaker visualization of the puncture needle on the puncture needle image. In FIG. 11, the ultrasound wave transmitted by the probe is perpendicular to the insertion direction of the puncture needle, that is, the angle θ is 90° and the angle β is an acute angle. Therefore, the reflection direction of the ultrasound wave 1 on the surface of the puncture needle is coincide with the transmitting direction of the ultrasound wave 1, that is, more ultrasound waves are reflected back to the probe 100, so that the puncture needle can be displayed in the puncture needle image more clearly. Therefore, in order to make the puncture needle to be displayed more clearly, after determining the optimized imaging parameter, the ultrasound imaging device 10 may transmit the first ultrasound wave to the puncture needle (that is, the interventional object) in at least one first angle according to the optimized imaging parameter, where the first ultrasound wave transmitted in the at least first angle may be perpendicular to the insertion direction of the puncture needle. For example, as shown in FIG. 12, the angle α between the insertion direction of the puncture needle and the surface of the probe is 45°, and in order to make the first ultrasound wave transmitted by the probe to be perpendicular to the insertion direction of the puncture needle as much as possible, the probe may transmit the first ultrasound wave to the puncture needle in the angle β=45°. Therefore, the first angle may be 45°. It should be noted that the probe may also transmit the first ultrasound wave in the 45° angle multiple times. Alternatively, the probe may transmit the first ultrasound wave in different angles such as 44.5°, 44.7°, 45° or 45.2°. Therefore, the first angle in which the ultrasound imaging device 10 transmits the first ultrasound wave through the probe 100 will not be limited herein.

In one embodiment, the transmission waveform of the first ultrasound wave may be a sine wave, a square wave or a triangular wave, etc. In addition, since the low-frequency wave has small attenuation, the first ultrasound wave may be a low frequency wave so as to obtain stronger ultrasound echoes.

After transmitting the first ultrasound wave to the interventional object in the at least one first angle according to the optimized imaging parameter, the ultrasound imaging device 10 may receive the first ultrasound echoes returned from the interventional object to obtain the first ultrasound echo data.

In step 204, the ultrasound image of the interventional object may be generated according to the first ultrasound echo data.

In this embodiment, the processor 105 may generate the ultrasound image of the interventional object according to the first ultrasound echo data.

In the embodiments of the present disclosure, the pulse echo detection technology may be used to obtain the ultrasound images. When the ultrasound waves propagate to the interfaces formed by different media, reflection and transmission will occur. Furthermore, since different human tissues or organs have different acoustic impedances and the ultrasound waves will propagate therein with different speeds, the ultrasound waves entering the human body will be reflected at the interface of different tissues or organs. The reflected echo data may be received by the probe, and be processed, so as to generate the ultrasound images. This technology is called pulse echo detection technology.

Therefore, after the ultrasound imaging device 10 obtains the first ultrasound echo data through the probe 100, the processor 105 may generate the ultrasound image of the interventional object according to the first ultrasound echo data, which may include the processor 105 performing the detection, amplification and interference removal processing, etc. on the first ultrasound echo data to generate the ultrasound image of the interventional object. It should be noted that the ultrasound image of the interventional object may be a two-dimensional or three-dimensional image, etc., which will not be limited herein.

In one embodiment, the ultrasound imaging device 10 may also perform denoising, analysis and inversion processing, etc. on the obtained first ultrasound echo data according to a preset mathematical model, and then perform a visualization processing on the processed first ultrasound echo data using computer image processing technology to generate the ultrasound image of the interventional object.

Therefore, in practical applications, there are many ways for generating the ultrasound image of the interventional object according to the first ultrasound echo data, which will not be limited herein.

In step 205. The ultrasound image of the target object may be obtained, and may be combined with the ultrasound image of the interventional object to obtain a combined image.

In this embodiment, the processor 105 may obtain the ultrasound image of the target object, and combine the ultrasound image of the target object with the ultrasound image of the interventional object to obtain the combined image.

The ultrasound imaging device 10 may obtain the ultrasound image of the target object so as to obtain the image of the tissue structures of the target object. The method for obtaining the ultrasound image of the target object may include the following steps.

In step 1, a third ultrasound wave may be transmitted to the target object in at least one second angle, and third ultrasound echoes returned from the target object may be received to obtain a third ultrasound echo data.

The ultrasound imaging device 10 may excite the probe 100 through the transmitting circuit 101 to transmit the third ultrasound wave to the target object in the at least one second angle, and control the probe 100 through the receiving circuit 103 to receive the third ultrasound echoes returned from the target object to obtain the third ultrasound echo data.

It should be noted that in step 1, the ultrasound imaging device 10 may, according to the optimized imaging parameter or the preset imaging parameter, transmit the third ultrasound wave to the target object in the at least one second angle and receive the third ultrasound echoes returned from the target object to obtain the third ultrasound echo data, which may be understood with reference to step 203 in FIG. 2 in which the ultrasound imaging device 10 transmits the first ultrasound wave to the interventional object in the at least a first angle according to the optimized imaging parameter and receive the first ultrasound echoes returned from the interventional object. The second angle may be an angle that is favorable for receiving the ultrasound echoes from the internal tissue of the target object. The second angle may be the angle between the direction in which the probe transmits the ultrasound wave to the target object and the direction perpendicular to the surface of the probe. It should be understood that this angle may be 0 degrees (that is, the ultrasound beam transmitted by the probe is perpendicular to the surface of the probe), or may be an acute angle.

In step 2, the B-mode ultrasound image of the target object may be generated according to the third ultrasound echo data.

The processor 105 may generate the B-mode ultrasound image of the target object according to the third ultrasound echo data.

In step 2, regarding the methods for the ultrasound imaging device 10 to generate the B-mode ultrasound image of the target object according to the third ultrasound echo data, reference may be made to step 204 in FIG. 2 where the ultrasound imaging device 10 generates the ultrasound image of the interventional object according to the first ultrasound echo data, which will not be described in detail here.

It should be noted that, although the ultrasound imaging device 10 obtains the ultrasound image of the interventional object in step 204 and obtains the ultrasound image of the target object in step 205, there is no sequence in these two processes. That is, the ultrasound image of the interventional object may be obtained first, or the ultrasound image of the target object may be obtained first, or they may be obtained at the same time, which will not be limited herein.

After obtaining the ultrasound image of the target object and the ultrasound image of the interventional object, the ultrasound imaging device 10 may combine the ultrasound image of the target object and the ultrasound image of the interventional object to obtain the combined image. In the embodiments of the present disclosure, the wavelet transformation method may be used to realize the combination of the ultrasound image of the target object with the ultrasound image of the interventional object. The wavelet transformation method is a time-scale analysis method for the signal, and has the ability to characterize the local characteristics of the signal in both time domain and frequency domain so as to obtain wavelet coefficients that characterize the similarity between the signal and the wavelet. It is a localized analysis in which the window size is fixed, but the window shape can be changed, and both the time window and the frequency domain window can be changed. Referring to FIG. 13 that is a schematic diagram of the image combination based on the wavelet transformation in one embodiment, obtaining the combined image using the wavelet transformation may include the following steps: 1) performing the discrete wavelet transformation (DWT) on the ultrasound image of the target object and the ultrasound image of the interventional object, respectively, to obtain the low-frequency component a1 and high-frequency component b1 corresponding to the ultrasound image of the target object and the low-frequency component a2 and the high-frequency component b2 corresponding to the ultrasound image of the interventional object; 2) fusing the low-frequency component a1 and the low-frequency component a2 according to the low-frequency fusion rule to obtain the low-frequency wavelet coefficient c1; 3) fusing the high-frequency component b1 and the high-frequency component b2 according to the high-frequency fusion rule to obtain the high-frequency wavelet coefficient c2; and 4) fusing the low-frequency wavelet coefficients c1 and the high-frequency wavelet coefficients c2 to obtain the wavelet coefficient, and performing the inverse discrete wavelet transformation (IDWT) on the wavelet coefficient obtained by the fusion (that is, performing image reconstruction) to obtain the fused image (that is, the combined image). Thereafter, the post-processing may be performed on the combined image. The post-processing may include adjusting the overall field gain uniformity of the combined image, enhancing the contrast of the combined image, highlighting the boundary and suppressing the noise of the combined image, or the like.

It should be noted that in the embodiments of the present disclosure, a transform domain fusion method, a pyramid method or other methods may also be used to obtain the combined image of the ultrasound image of the interventional object and the ultrasound image of the target object. Alternatively, the combined image of the ultrasound image of the interventional object and the ultrasound image of the target object may also be obtained by superimposing the ultrasound image of the interventional object with the ultrasound image of the target object, or by weighting and summing the ultrasound image of the interventional object and the ultrasound image of the target object, which will not be limited herein.

In the embodiments of the present disclosure, after obtaining the position information of the interventional object, the processor 105 may determine the optimized imaging parameter according to the position information. The first ultrasound wave may be transmitted to the interventional object according to the optimized imaging parameter to obtain the first ultrasound echo data, and the ultrasound image of the interventional object may be generated. The ultrasound image of the interventional object and the ultrasound image of the target object may be combined to obtain the combined image. Therefore, the operator will not need to adjust the parameters artificially to optimize the ultrasound image, and the surgical efficiency can be increased.

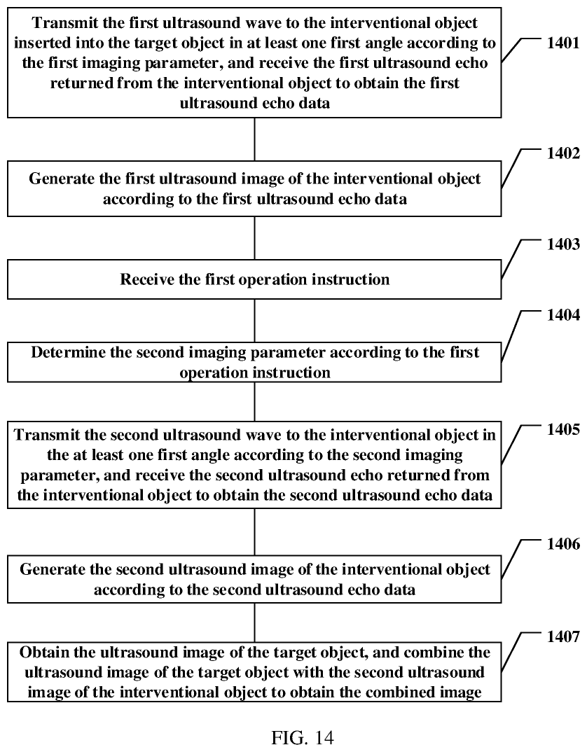

Referring to FIG. 14, another ultrasound imaging method may be provided, which may be applied in the ultrasound imaging device 10. The ultrasound imaging method may include the following steps.

In step 1401, the first ultrasound waves may be transmitted to the interventional object inserted into the target object in at least one first angle according to a first imaging parameter, and the first ultrasound echoes returned from the interventional object may be received to obtain the first ultrasound echo data.

In this embodiment, the ultrasound imaging device 10 may excite the probe 100 through the transmitting circuit 101 to transmit the first ultrasound to the interventional object inserted into the target object in the at least one first angle according to the first imaging parameter, and control the probe 100 to receive the first ultrasound echoes returned from the interventional object to obtain the first ultrasound echo data.

In this embodiment, regarding the process of the ultrasound imaging device 10 transmitting the first ultrasound wave to the interventional object inserted into the target object in the at least one first angle according to the first imaging parameter and receiving the first ultrasound echoes returned from the interventional object to obtain the first ultrasound echo data, reference may be made to the related description in step 203 shown in FIG. 2, and the details will not be described here.

The first imaging parameter may be an initial imaging parameter or a preset imaging parameter, etc., which will not be limited here.

In step 1402, a first ultrasound image of the interventional object may be generated according to the first ultrasound echo data.

In this embodiment, the processor 105 may generate the first ultrasound image of the interventional object according to the first ultrasound echo data.

In this embodiment, regarding step 1402, reference may be made to the related description in step 204 shown in FIG. 2, and the details will not be described here.

In step 1403, a first operation instruction may be received.

In this embodiment, the processor 105 may receive the first operation instruction. The first operation instruction may be an instruction that corresponds to the first operation and is generated by the user operating the ultrasound imaging device 10 through keys or touches. The first operation instruction may be used to trigger the ultrasound imaging device to optimize the displaying of the interventional object according to the position information of the interventional object. It should be noted that the first operation instruction may be sent by the operator through clicking a physical button on the ultrasound imaging device 10, or by the operator through clicking a display button on the touch display of the ultrasound imaging device.

In step 1404, a second imaging parameter may be determined according to the first operation instruction.

In this embodiment, the processor 105 may determine the second imaging parameter according to the first operation instruction.

In one embodiment, the processor 105 may obtain the position information of the interventional object in response to the first operation instruction, and determine the second imaging parameter according to the position information of the interventional object. In the embodiments of the present disclosure, the puncture needle is taken as an example of the interventional object. Therefore, the position information of the puncture needle may include the position of the needle tip. After obtaining the position information of the puncture needle, the second imaging parameter may be determined according to the position of the needle tip of the puncture needle.

Regarding the way for the ultrasound imaging device 10 to obtain the position information of the interventional object in step 1404, reference may be made to the related description of step 201 shown in FIG. 2 in which the ultrasound imaging device 10 obtains the position information of the interventional object, which will not be described in detail here.

After the ultrasound imaging device 10 obtains the position information of the interventional object, including the position of the needle tip of the puncture needle, the second imaging parameter may be determined according to the position of the needle tip of the puncture needle. It should be noted that, regarding the way for the ultrasound imaging device 10 to determine the second imaging parameter according to the position of the needle tip of the puncture needle in step 1404, reference may be made to the description of step 202 shown in FIG. 2 in which the ultrasound imaging device 10 determines the optimized imaging parameter according to the position information of the interventional object, which will not be described in detail here.

In step 1405, a second ultrasound wave may be transmitted to the interventional object in the at least one first angle according to the second imaging parameter, and the second ultrasound echoes returned from the interventional object may be received to obtain a second ultrasound echo data.

In this embodiment, the ultrasound imaging device 10 may excite the probe 100 through the transmitting circuit 101 to transmit the second ultrasound wave to the interventional object in the at least one first angle according to the second imaging parameter, and control the probe through the receiving circuit 103 to receive the second ultrasound echoes returned from the interventional object to obtain the second ultrasound echo data.

In step 1406, a second ultrasound image of the interventional object may be generated according to the second ultrasound echo data.

In this embodiment, the processor 105 may generate the second ultrasound image of the interventional object according to the second ultrasound echo data.

In this embodiment, regarding steps 1405 to 1406, reference may be made to related descriptions of step 203 to step 204 shown in FIG. 2, which will not be described in detail here.

In step 1407, the ultrasound image of the target object may be obtained, and may be combined with the second ultrasound image of the interventional object to obtain a combined image.

In this embodiment, the processor 105 may obtain the ultrasound image of the target object and combine the ultrasound image of the target object with the second ultrasound image of the interventional object to obtain the combined image.

In one embodiment, the processor 105 may excite the probe 100 through the transmitting circuit 101 to transmit the third ultrasound wave to the target object in at least one second angle, and control the probe 100 through the receiving circuit 103 to receive the third ultrasound echoes returned from the target object to obtain the third ultrasound echo data. The ultrasound image of the target object may be generated according to the third ultrasound echo data. The ultrasound image of the target object may be a B-mode ultrasound image.

In one embodiment, the processor 105 may excite the probe 100 through the transmitting circuit 101 to transmit the third ultrasound wave to the target object in the at least one second angle according to the second imaging parameter or the preset imaging parameter.

In this embodiment, regarding the way for the ultrasound imaging device 10 to obtain the ultrasound image of the target object in this step, reference may be made to the related description of the way for the ultrasound imaging device 10 to obtain the ultrasound image of the target object in step 205 shown in FIG. 2. Regarding the way for the ultrasound imaging device 10 to combine the ultrasound image of the target object with the second ultrasound image of the interventional object to obtain the combined image in step 1407, reference may be made to the description of the way for the ultrasound imaging device 10 to combine the ultrasound image of the target image with the ultrasound image of the interventional object to obtain the combined image.

In the embodiments of the present disclosure, it should be understood that the disclosed systems, devices and methods may be implemented in other ways. For example, the devices described above are only illustrative. For example, the division of the units is only a logical function division, and there may be other divisions in actual implementation. For example, multiple units or components may be combined or integrated into another system, or some features may be ignored or not implemented. In addition, the displayed or discussed mutual coupling or direct coupling or communication connection may be indirect coupling or communication connection through some interfaces, devices or units, and may be in electrical, mechanical or other forms.

The units described as separate components may or may not be physically separated, and the components displayed as units may or may not be physical units. They may be located in one place, or they may be distributed on multiple network units. Some or all of the units may be selected according to actual needs to achieve the objectives of the solutions of the embodiments.

In addition, the functional units in the embodiment of the present disclosure may be integrated into one processing unit, or each unit may exist alone physically, or two or more units may be integrated into one unit. The integrated unit may be implemented in the form of hardware or software functional unit.

If the integrated unit is implemented in the form of a software functional unit and sold or used as an independent product, it can be stored in a computer readable storage medium. The technical solutions of the present disclosure essentially, or the part that contributes to the existing technology, or all or part of the technical solutions, may be embodied in the form of a software product. The computer software product may be stored in a storage medium, and may include multiple instructions which may cause a computer device (which may be a personal computer, a server, or a network device, etc.) to perform all or part of the steps of the methods described in the embodiments of the present disclosure. The storage medium may include a U disk, a mobile hard disk, a read-only memory (ROM), a random access memory (RAM), a magnetic disk, an optical disk or other media that can store the program code.

The specific embodiments have been described above. However, the protection scope of the present disclosure will not be limited thereto. The modifications and replacements that are obvious for a person skilled in the art should all fall in the protection scope of the present disclosure. Therefore, the protection scope of the present disclosure should be determined by the claims.

1. An ultrasound imaging method, comprising:
transmitting, through a probe, a first ultrasound wave to an interventional object inserted into a target object in at least one first angle according to a first imaging parameter, and receiving, through the probe, a first ultrasound echo returned from the interventional object to obtain a first ultrasound echo data;
generating, through a processor, a first ultrasound image of the interventional object according to the first ultrasound echo data;
receiving a first operation instruction;
determining, through the processor, a second imaging parameter according to the first operation instruction;
transmitting, through the probe, a second ultrasound wave to the interventional object in the at least one first angle according to the second imaging parameter, and receiving, through the probe, a second ultrasound echo returned from the interventional object to obtain a second ultrasound echo data;
generating, through the processor, a second ultrasound image of the interventional object according to the second ultrasound echo data; and
obtaining an ultrasound image of the target object, and combining, through the processor, the ultrasound image of the target object with the second ultrasound image of the interventional object to obtain a combined image.

2. The method of claim 1, wherein determining, through the processor, the second imaging parameter according to the first operation instruction comprises:
obtaining a position information of the interventional object in response to the first operation instruction, wherein the position information comprises a position of a needle tip of a puncture needle; and
determining, through the processor, the second imaging parameter according to the position of the needle tip of the puncture needle.

3. The method of claim 1, wherein obtaining the ultrasound image of the target object comprises:
transmitting, through the probe, a third ultrasound wave to the target object in at least one second angle and receiving, through the probe, a third ultrasound echo returned from the target object to obtain a third ultrasound echo data; and
generating, through the processor, a B-mode ultrasound image of the target object according to the third ultrasound echo data.

4. The method of claim 3, wherein transmitting, through the probe, the third ultrasound wave to the target object in the at least one second angle and receiving the third ultrasound echo returned from the target object to obtain the third ultrasound echo data comprises:
transmitting, through the probe, the third ultrasound wave to the target object in the at least one second angle according to the second imaging parameter or a preset imaging parameter and receiving, through the probe, the third ultrasound echo returned from the target object to obtain the third ultrasound echo data.

5. An ultrasound imaging method, comprising:
obtaining a position information of an interventional object inserted into a target object;
determining, through a processor, an optimized imaging parameter according to the position information;
transmitting, through a probe, a first ultrasound wave to the interventional object in at least one first angle according to the optimized imaging parameter, and receiving, through the probe, a first ultrasound echo returned from the interventional object to obtain a first ultrasound echo data;
generating, through the processor, an ultrasound image of the interventional object according to the first ultrasound echo data; and
obtaining an ultrasound image of the target object, and combining, through the processor, the ultrasound image of the target object with the ultrasound image of the interventional object to obtain a combined image.

6. The method of claim 5, wherein obtaining the ultrasound image of the target object comprises:
transmitting, through the probe, a third ultrasound wave to the target object in at least one second angle and receiving, through the probe, a third ultrasound echo returned from the target object to obtain a third ultrasound echo data; and
generating, through the processor, a B-mode ultrasound image of the target object according to the third ultrasound echo data.

7. The method of claim 6, wherein transmitting, through the probe, the third ultrasound wave to the target object in the at least one second angle and receiving the third ultrasound echo returned from the target object to obtain the third ultrasound echo data comprises:
transmitting, through the probe, the third ultrasound wave to the target object in the at least one second angle according to the optimized imaging parameter or a preset imaging parameter and receiving, through the probe, the third ultrasound echo returned from the target object to obtain the third ultrasound echo data.

8. The method of claim 5, wherein the position information comprises a position of a needle tip of a puncture needle.

9. The method of claim 8, wherein obtaining the position information of the interventional object inserted into the target object comprises:
detecting, through a magnetic field sensor, a magnetic induction intensity generated by the puncture needle that is magnetized; and
determining, through the processor, the position of the needle tip of the puncture needle according to the magnetic induction intensity.

10. The method of claim 5, wherein the optimized imaging parameter comprises at least one of an ultrasound scanning range, an ultrasound scanning depth, and a position of a focus of ultrasound wave.

11. The method of claim 8, wherein determining, through the processor, the optimized imaging parameter according to the position information comprises:
determining, through the processor, a position of a focus of the first ultrasound wave according to the position of the needle tip of the puncture needle, wherein the needle tip of the puncture needle is within a range of the focus of the first ultrasound wave.

12. The method of claim 8, wherein determining, through the processor, the optimized imaging parameter according to the position information comprises:
determining, through the processor, a scanning range of the first ultrasound wave according to the position of the needle tip of the puncture needle, wherein a distance from the position of the needle tip of the puncture needle to a longitudinal boundary of a display area of the ultrasound image of the target object meets a first preset condition.

13. The method of claim 8, wherein determining, through the processor, the optimized imaging parameter according to the position information comprises:
determining, through the processor, a scanning depth of the first ultrasound wave according to the position of the needle tip of the puncture needle, wherein a distance from the position of the needle tip of the puncture needle to a horizontal boundary of a display area of the ultrasound image of the target object meets a second preset condition.

14. A non-transitory computer readable storage medium storing multiple instructions, wherein the multiple instructions, when being executed by a computer, cause the computer to:
transmitting, through a probe, a first ultrasound wave to an interventional object inserted into a target object in at least one first angle according to a first imaging parameter, and receiving, through the probe, a first ultrasound echo returned from the interventional object to obtain a first ultrasound echo data;
generating a first ultrasound image of the interventional object according to the first ultrasound echo data;
receiving a first operation instruction;
determining a second imaging parameter according to the first operation instruction;
transmitting, through the probe, a second ultrasound wave to the interventional object in the at least one first angle according to the second imaging parameter, and receiving, through the probe, a second ultrasound echo returned from the interventional object to obtain a second ultrasound echo data;
generating a second ultrasound image of the interventional object according to the second ultrasound echo data; and
obtaining an ultrasound image of the target object, and combining the ultrasound image of the target object with the second ultrasound image of the interventional object to obtain a combined image.
